(19) United States
(12) Patent Application Publication
(10) Pub. No.: US 2009/0312556 A1
(43) Pub. Date: Dec. 17, 2009

(54) N-(2-AMINIPHENYL)-4-(5-((ETHYLAMINO)METHYL)-1,2-THIAZOL-2-YL) BENZAMIDE AND N-(2-AMINOPHENYL)-4-(5-((ISOPROPYLAMINO)METHYL)-1,2-THIAZOL-2-YL) BENZAMIDE AS HDAC INHIBITORS

(75) Inventors: Elaine Sophie, Elizabeth Stokes, Macclesfield (GB); Gregory Carr, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA R& D ALDERLY, Macclesfield (GB)

(21) Appl. No.: 11/721,605

(22) PCT Filed: Dec. 15, 2005
(86) PCT No.: PCT/GB05/04856

§ 371 Date: Jun. 13, 2007

(30) Foreign Application Priority Data
Dec. 18, 2004 (GB) 0427758.8

(51) Int. Cl. C07D 277/30

(52) U.S. Cl. 548/204

(57) ABSTRACT

The invention concerns novel benzamide compounds of Formula (I): wherein R wherein is ethyl or isopropyl; or a pharmaceutically acceptable salt or pro-drug form thereof. The invention also concerns processes for the preparation of such compounds, pharmaceutical compositions containing them and their use in the manufacture of a medicament for use as an antiproliferative agent in the prevention or treatment of tumours or other proliferative conditions, which are sensitive to the inhibition of histone deacetylase (HDAC).

This invention concerns certain novel benzamide compounds, or pharmaceutically acceptable salts or pro-drug forms thereof, which are potent inhibitors of the enzyme histone deacetylase (HDAC) and are accordingly of value for the treatment of a number of disease states in a warm-blooded animal, such as man, in which HDAC activity is implicated. Examples of such disease states include cancer (Marks et al., Nature Reviews, 1, 194-202, (2001)), cystic fibrosis (Li, S. et al, J. Biol. Chem., 274, 7803-7815, (1999)), Huntingdons chorea (Steffan, J. S. et al., Nature, 413, 739-743, (2001)) and sickle cell anemia (Gabbianelli, M. et al., Blood, 95, 3555-3561, (2000)) and accordingly, the benzamide compounds of the present invention are useful in methods of treating any of these particular disease states. The invention also concerns processes for the manufacture of these novel benzamide compounds, to pharmaceutical compositions containing them and to their use in therapeutic methods, for example in the manufacture of medicaments to inhibit HDAC in a warm-blooded animal, such as man.

In the eukaryotic cell, DNA is routinely compacted to prevent transcription factor accessibility. When the cell is activated this compacted DNA is made available to DNA-binding proteins, thereby allowing the induction of gene transcription (Beato, M., J. Med. Chem., 74, 711-724 (1996); Wolffe, A. P., Nature, 387, 16-17 (1997)). Nuclear DNA associates with nuclear proteins known as histones to form a complex called chromatin. The core histones, termed H2A, H2B, H3 and H4, are surrounded by 146 base pairs of DNA to form the fundamental unit of chromatin, and which is known as the nucleosome. The N-terminal tails of the core histones contain lysine residues that are sites for post-transcriptional acetylation. Acetylation of the terminal amino group on the lysine side chain neutralizes the potential of the side chain to form a positive charge, and is thought to impact on chromatin structure.

Histone Deacetylases (HDACs) are zinc-containing enzymes which catalyse the removal of acetyl groups from the ε-amino termini of lysine residues clustered near the amino terminus of nucleosomal histones. HDACs may be divided into two classes, the first (HDAC 1, 2, 3 and 8) represented by yeast Rpd3-like proteins, and the second (HDAC 4, 5, 6, 7, 9 and 10) represented by yeast Hda1-like proteins. The reversible process of acetylation is known to be important in transcriptional regulation and cell-cycle progression. In addition, HDAC deregulation has been associated with several cancers and HDAC inhibitors, such as Trichostatin A (a natural product isolated from Streptomyces hygroscopicus), have been shown to exhibit significant cell growth inhibition and anti-tumour effects (Meinke, P. T., Current Medicinal Chemistry, 8, 211-235 (2001)). Yoshida et al, (Exper. Cell Res., 177, 122-131 (1988)) teach that Trichostatin A causes the arrest of rat fibroblasts at the G1 and G2 phases of the cell cycle, thereby implicating the role of HDAC in the regulation of the cell cycle. Furthermore, Trichostatin A has been shown to induce terminal differentiation, inhibit cell growth, and prevent the formation of tumours in mice (Finnin et al., Nature, 401, 188-193 (1999)).

It is known from the published International Patent Application Numbers WO 03/087057 and WO 03/092686 that certain benz amide derivatives are inhibitors of HDAC. One particular compound disclosed in WO 03/087057 is N-(2-aminophenyl)-4-[5-(piperidin-1-ylmethyl)-1,3-thiazol-2-yl]benzamide, the structure of which is shown below.

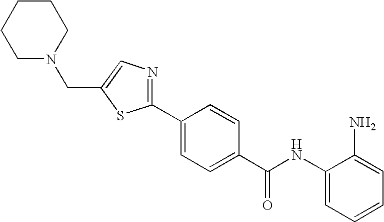

We have now surprisingly found that certain benzamide derivatives that comprise certain substituted-aminomethyl substituent groups in the 5-position of the 1,3-thiazol-2-yl moiety are potent inhibitors of HDAC. We have also found that these derivatives additionally possess a number of other favourable pharmaceutical properties, including advantageous cell and in-vivo potencies, and/or advantageous DMPK properties (for example, a favourable bioavailability profile and/or favourable free-plasma levels and/or a favourable half life and/or a favourable volume of distribution), and moderate to low activities in a hERG assay.

According to the present invention there is provided a compound of formula (I):

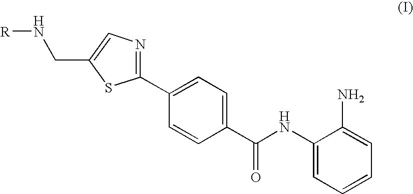

wherein R is ethyl or isopropyl;

or a pharmaceutically acceptable salt or pro-drug form thereof.

Preferably, R is ethyl.

Particular compounds of the invention are:
N-(2-aminophenyl)-4-{5-[(ethylamino)methyl]-1,3-thiazol-2-yl}benzamide; and
N-(2-aminophenyl)-4-{5-[(isopropylamino)methyl]-1,3-thiazol-2-yl}benzamide.

It is to be understood that certain compounds of the Formula I may exist in solvated as well as unsolvated forms such as, for example, hydrated forms. It is to be understood that the invention encompasses all such solvated forms which possess antiproliferative activity.

It is also to be understood that certain compounds of the Formula I may exhibit polymorphism, and that the invention encompasses all such forms which possess antiproliferative activity.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

The compounds of the invention may be administered in the form of a pro-drug—that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable amide derivatives that may be formed at an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents:—

a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988);

f) N. Kakeya, et al., Chem. Pharm. Bull., 32, 692 (1984);

### g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

A suitable pharmaceutically-acceptable pro-drug of a compound of the Formula I is, for example, an in vivo cleavable amide derivative thereof. Suitable pharmaceutically-acceptable amides formed from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

Another aspect of the present invention provides a process for preparing a compound of formula (I) or a pharmaceutically acceptable salt or pro-drug form thereof (wherein R is, unless otherwise specified, as hereinbefore defined), said process comprising the steps of:
(a) the reaction, in the presence of a suitable base, of a compound of the formula (II)

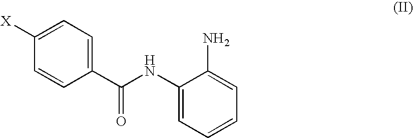

wherein X is a reactive group, with an organometallic compound of the formula (III)

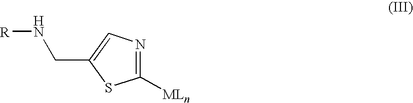

wherein M is a metal, L is a ligand and n is 0 to 3;
(b) The reaction, in the presence of a suitable base, of a compound of the formula (IV)

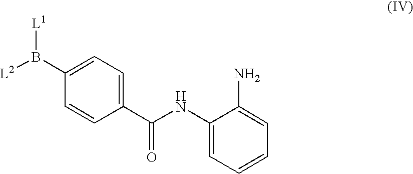

wherein L¹ and L² are ligands, with a compound of the formula (V)

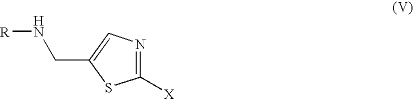

wherein X is a reactive group; or

(c) the reaction, in the presence of 4-(4,6-dimethoxy-1,3,5-triazinyl-2-yl)-4-methylmorpholinium chloride and a suitable base, of a compound of the formula (VI)

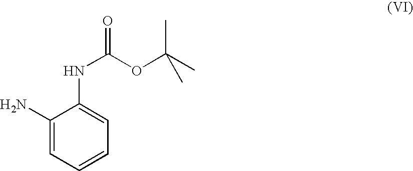

with a compound of the formula (VII);

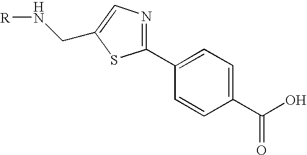

or

(d) the reaction, in the presence of a suitable base, of a compound of formula (VIII)

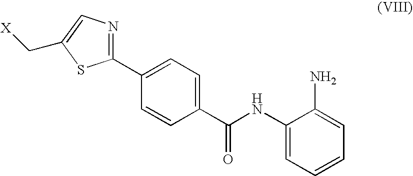

wherein X is a reactive group, with a compound of formula (IX);

R—NH₂ (IX)
or

(e) the reaction, in the presence of a suitable reducing agent and an acid, of a compound of formula (X)

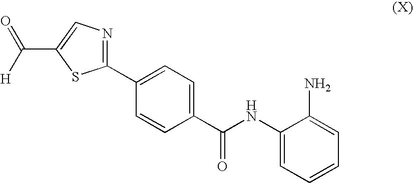

with a compound of formula (IX);

R—NH₂ (IX)

and thereafter, if necessary, removing any protecting groups.

A suitable base for process (a), (b), (c) or (d) is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal hydride, for example sodium hydride, or a alkaline earth metal bicarbonate such as sodium bicarbonate, or an alkaline earth metal hydrogencarbonate such as sodium hydrogencarbonate, or a metal alkoxide such as sodium ethoxide.

A suitable reactive group X is, for example, a halo, alkoxy, aryloxy or sulphonyloxy group, for example a chloro, bromo, methoxy, phenoxy, methanesulphonyloxy, trifluoromethanesulphonyloxy or toluene-4-sulphonyloxy group. The reactions are conveniently carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran, 1,2-dimethoxyethane or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reactions are conveniently carried out at a temperature in the range, for example, 10 to 250° C., preferably in the range 40 to 80° C.;

Metal M may be any metal that is known in the literature to form organometallic compounds that undergo catalytic cross coupling reactions. Examples of suitable metals include boron, tin, zinc, magnesium.

The value of integer n will depend on the metal M.

Suitable values for the ligand L, when present, include, for example, a hydroxy, a halo, (1-4C)alkoxy or (1-6C)alkyl ligand, for example a hydroxy, bromo, chloro, fluoro, iodo, methoxy, ethoxy, propoxy, isopropoxy, butoxy, methyl, ethyl, propyl, isopropyl or butyl ligand.

A suitable value for the ligands L¹ and L² which are present on the boron atom include, for example, a hydroxy, (1-4C)alkoxy or (1-6C)alkyl ligand, for example a hydroxy, methoxy, ethoxy, propoxy, isopropoxy, butoxy, methyl, ethyl, propyl, isopropyl or butyl ligand. Alternatively the ligands L¹ and L² may be linked such that, together with the boron atom to which they are attached, they form a ring. For example, L¹ and L² together may define an oxy-(2-4C)alkylene-oxy group, for example an oxyethyleneoxy or oxytrimethyleneoxy group such that, together with the boron atom to which they are attached, they form a cyclic boronic acid ester group;

A suitable catalyst for process (a) or (b) above includes, for example, a metallic catalyst such as a palladium(0), palladium(II), nickel(0) or nickel(II) catalyst, for example tetrakis(triphenylphosphine)palladium(0), palladium(II) chloride, palladium(II) bromide, bis(triphenylphosphine)palladium(II) chloride, tetrakis(triphenylphosphine)nickel(0), nickel(II) chloride, nickel(II) bromide, bis(triphenylphosphine)nickel(II) chloride or dichloro[1-1′-bis(diphenylphosphino)ferrocene]palladium(II). In addition a free radical initiator may conveniently be added, for example an azo compound such as azo(bisisobutyronitrile);

A suitable reducing agent for process (e) includes, for example, an inorganic borohydride salt such as, sodium borohydride, sodium triacetoxyborohydride or sodium cyanoborohydride.

A suitable acid for process (e), includes a Bronsted acid such as, for example formic acid, acetic acid, trifluoroacetic acid, hydrochloric acid, sulphuric acid, paratoluene sulfonic acid or camphor sulfonic acid; or a Lewis acid of formula MX_{z}, wherein M is a metal, X is a reactive group as herein defined and the value of z is 1-6 and will depend on the metal M. Typical examples of suitable Lewis acids include boron trifluoride, scandium(III) trifluoromethanesulfonate, tin (VI) chloride, titanium (IV) isopropoxide or zinc (II) chloride.

Suitably, the intermediate compound (VII)

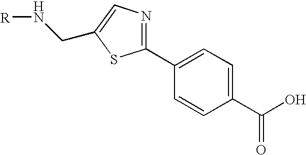

is prepared by a process (process (f)) which comprises the steps of:

(i) reacting a compound of formula (XII)

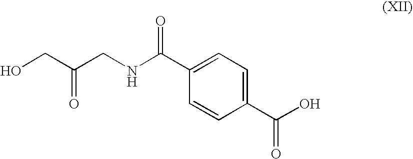

with a suitable thionating agent to produce a compound of formula (XIII)

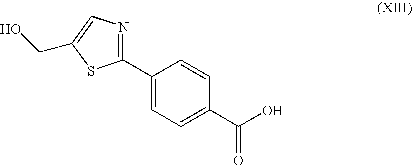

(ii) coupling the compound of formula (IX)

R—NH₂ (IX)

with the compound of Formula (XIII) prepared in step (i) above;

and thereafter, if necessary, removing any protecting groups.

The compound of formula (XIII) may be coupled to the compound of Formula (IX) by any suitable means known in the art. For example, the reaction of the compound of formula (XIII) with methane sulfonyl chloride, in the presence of a base, followed by further reaction of the resultant product with a compound of formula (IX); or reaction of the compound of formula (XIII) with an oxidising agent, such as Dess Martin periodinane to produce a compound of formula (XIV)

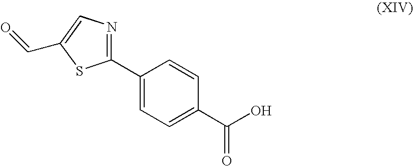

and then reacting said compound with a compound of formula (IX) in the presence of a suitable reducing agent and an acid to produce a compound of formula (VII).

Suitably the intermediate compound of formula (VIII),

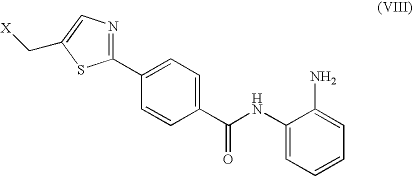

wherein X is a leaving group as hereinbefore defined, is prepared by a process (process (g)), which comprises the steps of:
(i) reacting a compound of formula (XII)

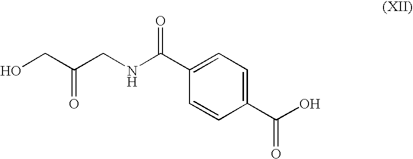

with a suitable thionating agent to produce a compound of formula (XIII)

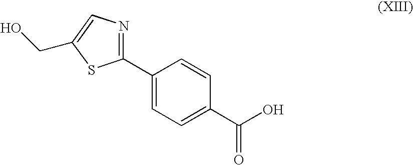

(ii) reacting the compound of the formula (XIII) prepared in step (i) above with a compound of formula (VI),

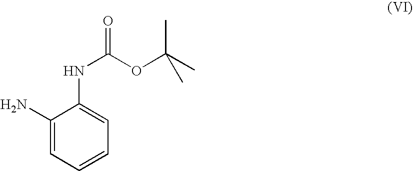

in the presence of 4-(4,6-dimethoxy-1,3,5-triazinyl-2-yl)-4-methylmorpholinium chloride;
and thereafter (in any suitable order):
(a) converting the hydroxyl group of the hydroxymethyl substituent group attached to the 5-position of the 1,3-thiazol-2-yl moiety to a leaving group X as hereinbefore defined; and
(b) removing any protecting groups.

Any suitable method known in the art for converting the hydroxyl group of the hydroxymethyl substituent group attached to the 5-position of the 1,3-thiazol-2-yl moiety to a leaving group X may be used, and such methods will be known to persons skilled in the art.

Suitably the intermediate compound of formula (X)

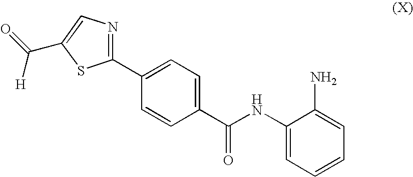

is prepared by a process (process (h)), which comprises the steps of:
(i) reacting a compound of formula (XII)

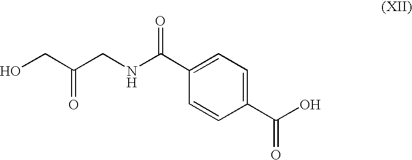

with a suitable thionating agent to produce a compound of formula (XIII)

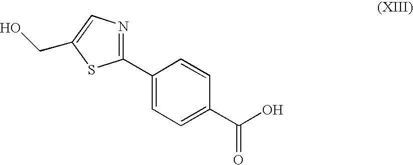

(ii) reacting the compound of the formula (XIII) prepared in step (i) above with a compound of formula (VI),

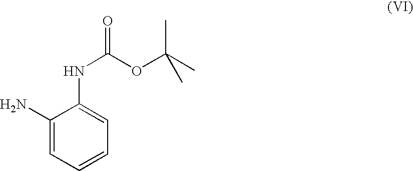

in the presence of 4-(4,6-dimethoxy-1,3,5-triazinyl-2-yl)-4-methylmorpholinium chloride;
and thereafter (in any suitable order):
(a) oxidising the hydroxy group of the hydroxymethyl group attached to the 5-position of the 1,3-thiazol-2-yl moiety to a carbaldehyde group; and
(b) removing any protecting groups.

Any suitable method known in the art for oxidising the hydroxy group of the hydroxymethyl group attached to the 5-position of the 1,3-thiazol-2-yl moiety to a carbaldehyde group may be used, and such methods will be known to persons skilled in the art.

Suitable thionating agents for use in either of processes (f), (g) and (h) above includes, for example, an organic compound of phosphorus and sulfur such as phosphorus trisulfide, phosphorus pentasulfide or 2,4-bis(4-methoxyphenyl)-1,3-dithia-2,4-diphosphetane-2,4-disulfide (Lawesson's reagent).

It will also be appreciated that in some of the reactions mentioned herein it may be necessary or desirable to protect any sensitive groups in the compounds. The instances where protection is necessary or desirable and suitable methods for protection are known to those skilled in the art. Conventional protecting groups may be used in accordance with standard practice (for illustration see T. W. Green & P. G. M. Wuts, Protective Groups in Organic Synthesis, 3^{rd} Edition, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

### Biological Assays

The following assays can be used to measure the effects of the compounds of the present invention as HDAC inhibitors, as inhibitors in vitro of recombinant human HDAC1 produced in Hi5 insect cells, and as inducers in vitro & in vivo of Histone H3 acetylation in whole cells and tumours. They also assess the ability of such compounds to inhibit proliferation of human tumour cells.

### (a) In Vitro Enzyme Assay of Recombinant HDAC1

HDAC inhibitors were screened against recombinant human HDAC1 produced in Hi5 insect cells. The enzyme was cloned with a FLAG tag at the C-terminal of the gene and affinity purified using Anti-FLAG M2 agarose from SIGMA (A2220).

The deacetylase assays were carried out in a 50 μl reaction. HDAC1 (75 ng of enzyme) diluted in 15 μl of reaction buffer (25 mM TrisHCl (pH 8), 137 mM NaCl, 2.7 mM KCl, 1 mM MgCl₂) was mixed with either buffer alone (10 μl) or buffer containing compound (10 μl) for 30 minutes at ambient temperature. 25 μM acetylated histone H4 peptide (KI 174 Biomol) diluted in 25 μl of buffer was then added to the reaction and incubated for one hour at ambient temperature. The reaction was stopped by addition of an equal volume (50 μl) of Fluor de Lys developer (Biomol) containing Trichostatin A at 2 μM. The reaction was allowed to develop for 30 minutes at ambient temperature and then fluorescence measured at an excitation wavelength of 360 nM and an emission wavelength of 465 nM. IC₅₀ values for HDAC enzyme inhibitors were determined by performing dose response curves with individual compounds and determining the concentration of inhibitor producing fifty percent decrease in the maximal signal (diluent control).

### (b) In Vitro Assay of Inhibition of Proliferation in Whole Cells

Inhibition of proliferation in whole cells was assayed using the Promega cell titer 96 aqueous proliferation assay (Promega #G5421). HCT116 cells were seeded in 96 well plates at 1×10³ cells/well, and allowed to adhere overnight. They were treated with inhibitors for 72 hours. 20 μl of the tetrazolium dye MTS was added to each well and the plates were re-incubated for 3 hours. Absorbance was then measured on a 96 well plate reader at 490 nM. The IC₅₀ values for HDAC inhibitors were determined by performing dose response curves with individual compounds and determining the concentration of inhibitor producing fifty percent decrease in the maximal signal (diluent control).

### (c) In Vitro Enzyme Assay of Histone Deacetylase Activity in Whole Cells

Histone H3 acetylation in whole cells was measured using immunohistochemistry and analysis using the Cellomics arrayscan. A549 or HCT116 cells were seeded in 96 well plates at 1×10⁴ cells/well, and allowed to adhere overnight. They were treated with inhibitors for 24 hours and then fixed in 1.8% formaldehyde in tris buffered saline (TBS) for one hour. Cells were permeabilized with ice-cold methanol for 5 minutes, rinsed in TBS and then blocked in TBS 3% low-fat dried milk for 90 minutes. Cells were then incubated with polyclonal antibodies specific for the acetylated histone H3 (Upstate #06-599) diluted 1 in 500 in TBS 3% milk for one hour. Cells were rinsed three times in TBS and then incubated with fluorescein conjugated secondary antibodies (Molecular Probes #A11008) & Hoechst 333542 (1 μg/ml) (Molecular Probes #H3570) in TBS plus 1% Bovine serum albumin (Sigma #B6917) for one hour. Unbound antibody was removed by three rinses with TBS and after the final rinse 100 μl of TBS was added to the cells and the plates sealed and analysed using the Cellomics arrayscan.

EC₅₀ values for HDAC inhibitors were determined by performing dose response curves with individual compounds and then determining the concentration of inhibitor producing fifty percent of the maximal signal (reference compound control—Trichostatin A (Sigma)).

### (d) In Vivo Assay of Histone Deacetylase Activity in Tumours and Normal Tissues

HDAC inhibitors were screened for in vivo blood levels and efficacy.

Male Hans Wistar rats were dosed orally with HDAC inhibitors at 25 mg/kg using a gavage in a formulation of 0.5% w/v Methocel+0.1% w/v Poly (HPMC/Tween). At 6, 12 and 20 hrs after the dose, rats were terminated and spleens removed. Spleens were snap frozen in liquid nitrogen and then stored at −80° C. until analysis.

Excised tissues (stored at −80° C.) were disaggregated manually and homogenised using a glass homogeniser in buffer (25 mM Hepes Ph7.6, 120 mM NaCl, 5 mM beta-glycerophosphate, 1 mM MgCl, 0.2 mM EDTA, 1 mM EGTA). Cells were further disrupted by sonication and DNA digested using 300 u/ml benzonase (Sigma #E1014). Cell lysates were assayed for protein concentration using the Pierce BCA Protein kit (#23227) and 5 μg protein samples were separated using SDS PAGE gels. Proteins were then transferred onto Nitroclleulose membrane. The membranes were probed with Upstate 06-599 anti acetyl H3 histone antibody ( 1/4000) and secondarily probed for by DAKO p0448 anti rabbit HRP conjugated antibody ( 1/2000). The signal was detected by Pierce 34075 Super Signal West Dura chemiluminescence and quantified on the Syngene Chemigenius system. Results are normalized relative to a comparator compound, N-(2-aminophenyl)-4-[5-(piperidin-1-ylmethyl)-1,3-thiazol-2-yl]benzamide (disclosed in International Patent Publication No. WO 03/087057), which is assigned the value of 100%.

(e) hERG-Encoded Potassium Channel Inhibition Assay

This assay determines the ability of a test compound to inhibit the tail current flowing through the human ether-a-go-go-related-gene (hERG)-encoded potassium channel.

Human embryonic kidney (HEK) cells expressing the hERG-encoded channel were grown in Minimum Essential Medium Eagle (EMEM; Sigma-Aldrich catalogue number M2279), supplemented with 10% Foetal Calf Serum (Labtech International; product number 4-101-500), 10% M1 serum-free supplement (Egg Technologies; product number 70916) and 0.4 mg/ml Geneticin G418 (Sigma-Aldrich; catalogue number G7034). One or two days before each experiment, the cells were detached from the tissue culture flasks with Accutase is (TCS Biologicals) using standard tissue culture methods. They were then put onto glass coverslips resting in wells of a 12 well plate and covered with 2 ml of the growing media.

For each cell recorded, a glass coverslip containing the cells was placed at the bottom of a Perspex chamber containing bath solution (see below) at room temperature (˜20° C.). This chamber was fixed to the stage of an inverted, phase-contrast microscope. Immediately after placing the coverslip in the chamber, bath solution was perfused into the chamber from a gravity-fed reservoir for 2 minutes at a rate of ˜2 ml/min. After this time, perfusion was stopped.

A patch pipette made from borosilicate glass tubing (GC120F, Harvard Apparatus) using a P-97 micropipette puller (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configuration of the patch clamp technique. Following “break-in”, which was done at a holding potential of −80 mV (set by the amplifier), and appropriate adjustment of series resistance and capacitance controls, electrophysiology software (Clampex, Axon Instruments) was used to set a holding potential (−80 mV) and to deliver a voltage protocol. This protocol was applied every 15 seconds and consisted of a 1 s step to +40 mV followed by a Is step to −50 mV. The current response to each imposed voltage protocol was low pass filtered by the amplifier at 1 kHz. The filtered signal was then acquired, on line, by digitizing this analogue signal from the amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |

| Parameter | Pipette | Bath |
|---|---|---|
| pH | 7.18-7.22 | 7.40 |
| pH adjustment with | 1M KOH | 1M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hill equation using a standard data-fitting package. If the level of inhibition seen at the highest test concentration did not exceed 50%, no potency value was produced and a percentage inhibition value at that concentration was quoted.

Although the pharmacological properties of the compounds of the Formula I vary with structural change as expected, in general activity possessed by compounds of the Formula I, may be demonstrated at the following concentrations or doses in one or more of the above tests (a), (b), (c) and (d):—
Test (a):—IC₅₀ in the range, for example, 100 nM or less;
Test (b):—IC₅₀ in the range, for example, 0.5 μM or less;
Test (c):—IC₅₀ in the range, for example, 0.5 μM or less;
Test (d):—greater than 120% of the activity of the comparator compound, N-(2-aminophenyl)-4-[5-(piperidin-1-ylmethyl)-1,3-thiazol-2-yl]benzamide (which is assigned the value of 100%) at 12 hours after dosing;

No physiologically unacceptable toxicity was observed in Test (d) at the effective dose for compounds tested of the present invention. Accordingly no untoward toxicological effects are expected when a compound of Formula I, or a pharmaceutically-acceptable salt thereof, as defined hereinbefore is administered at the dosage ranges defined hereinafter.

By way of example, using Test (a) for the inhibition of HDAC1 and Test (b) for the inhibition of proliferation in whole cells, the compound described in Example 2 herein gave the IC₅₀ results shown below in Table A below:

TABLE A

| | | IC₅₀ (nM) Test (b) |
|---|---|---|
| | IC₅₀ (nM) Test (a) | (In vitro assay for the |
| | (In vitro assay for the | inhibition of whole cell |
| Compound of Example | inhibition of HDAC1) | proliferation) |
| 1 | 29 | 183 |

According to a further aspect of the invention there is provided a pharmaceutical composition which comprises a compound of the formula (I), or a pharmaceutically acceptable salt or pro-drug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective cell cycle inhibitors (anti-cell proliferation agents), and this property is believed to arise from their HDAC inhibitory activity. We also believe that the compounds of the present invention may be involved in the inhibition of angiogenesis, activation of apoptosis and differentiation. Accordingly the compounds of the present invention are expected to be useful in the treatment of diseases or medical conditions mediated alone or in part by HDAC enzymes, i.e. the compounds may be used to produce a HDAC inhibitory effect in a warm-blooded animal in need of such treatment. Thus, the compounds of the present invention provide a method for treating the proliferation of malignant cells characterised by inhibition of HDAC enzymes, i.e. the compounds may be used to produce an anti-proliferative effect mediated alone or in part by the inhibition of HDACs.

According to another aspect of the present invention there is provided a compound of the formula (I), or a pharmaceutically acceptable salt or pro-drug thereof, as defined hereinbefore for use in a method of treatment of the human or animal body by therapy.

Thus according to a further aspect of the invention there is provided a compound of the formula (I), or a pharmaceutically acceptable salt or pro-drug thereof, as defined hereinbefore for use as a medicament.

According to a further aspect of the invention there is provided the use of a compound of the formula (I), or a pharmaceutically acceptable salt or pro-drug thereof, as defined hereinbefore in the manufacture of a medicament for use in the production of a HDAC inhibitory effect in a warm-blooded animal such as man.

According to a further feature of this aspect of the invention there is provided a method for producing a HDAC inhibitory effect in a warm-blooded animal, such as man, in need of such treatment which comprises administering to said animal an effective amount of a compound of the formula (I), or a pharmaceutically acceptable salt or pro-drug thereof, as defined hereinbefore.

According to a further aspect of the invention there is provided the use of a compound of the formula (I), or a pharmaceutically acceptable salt or pro-drug thereof, as defined hereinbefore in the manufacture of a medicament for use in the production of a cell cycle inhibitory (anti-cell-proliferation) effect in a warm-blooded animal such as man.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound of the formula (I), or a pharmaceutically acceptable salt or pro-drug thereof, as defined hereinbefore.

According to an additional feature of this aspect of the invention there is provided a method of treating cancer in a warm-blooded animal, such as man, in need of such treatment which comprises administering to said animal an effective amount of a compound of the formula (I), or a pharmaceutically acceptable salt or pro-drug thereof, as defined hereinbefore.

According to a further feature of the invention there is provided a compound of the formula (I), or a pharmaceutically acceptable salt or pro-drug thereof, as defined hereinbefore in the manufacture of a medicament for use in the treatment of cancer.

According to an additional feature of this aspect of the invention there is provided a compound of the formula (I), or a pharmaceutically acceptable salt or pro-drug thereof, or a pharmaceutically acceptable salt thereof, as defined hereinbefore, for use in the treatment of cancer.

In a further aspect of the present invention there is provided the use of a compound of the formula (I), or a pharmaceutically acceptable salt or pro-drug thereof, as defined hereinbefore, in the manufacture of a medicament for use in lung cancer, colorectal cancer, breast cancer, prostate cancer, lymphoma and/or leukaemia.

In a further aspect of the present invention there is provided a method of treating lung cancer, colorectal cancer, breast cancer, prostate cancer, lymphoma or leukaemia, in a warm-blooded animal, such as man, in need of such treatment which comprises administering to said animal an effective amount of a compound of the formula (I), or a pharmaceutically acceptable salt or pro-drug thereof, as defined hereinbefore.

Cancers that are amenable to treatment with the present invention include oesophageal cancer, myeloma, hepatocellular, pancreatic and cervical cancer, Ewings tumour, neuroblastoma, kaposis sarcoma, ovarian cancer, breast cancer, colorectal cancer, prostate cancer, bladder cancer, melanoma, lung cancer [including non small cell lung cancer (NSCLC) and small cell lung cancer (SCLC)], gastric cancer, head and neck cancer, brain cancer, renal cancer, lymphoma and leukaemia.

The HDAC inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antiestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αᵥβ₃ function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies;
(x) Cell cycle inhibitors including for example CDK inhibitors (e.g. flavopiridol) and other inhibitors of cell cycle checkpoints (e.g. checkpoint kinase); inhibitors of aurora kinase and other kinases involved in mitosis and cytokinesis regulation (e.g. mitotic kinesins); and other histone deacetylase inhibitors; and
(xi) differentiation agents (for example retinoic acid and vitamin D).

According to this aspect of the invention there is provided a pharmaceutical composition comprising a compound of the formula (I) as defined hereinbefore and an additional anti-tumour substance as defined hereinbefore for the conjoint treatment of cancer.

There is further provided is a compound of the formula (I), or a pharmaceutically acceptable salt or pro-drug thereof, as defined hereinbefore, for use in a method of treating inflammatory diseases, autoimmune diseases and allergic/atopic diseases.

In particular a compound of the formula (I), or a pharmaceutically acceptable salt thereof, as defined hereinbefore, is provided for use in a method of treating inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is a compound of the formula (I), or a pharmaceutically acceptable salt or pro-drug thereof, as defined hereinbefore, for use as a medicament in the treatment of inflammatory diseases, autoimmune diseases and allergic/atopic diseases in a warm-blooded animal such as man.

In particular a compound of the formula (I), or a pharmaceutically acceptable salt or pro-drug thereof, as defined hereinbefore, is provided for use as a medicament in the treatment of inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is the use of a compound of the formula (I), or a pharmaceutically acceptable salt thereof, as defined hereinbefore, in the manufacture of a medicament for use in the treatment of inflammatory diseases, autoimmune diseases and allergic/atopic diseases in a warm-blooded animal such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula (I) and their pharmaceutically acceptable salts thereof, are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

The invention will now be illustrated in the following Examples in which, generally:

(i) operations were carried out at ambient temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) column chromatography (by the flash procedure) and medium pressure liquid chromatography (MPLC) were performed on Merck Kieselgel silica (Art. 9385) or Merck Lichroprep RP-18 (Art. 9303) reversed-phase silica obtained from E. Merck, Darmstadt, Germany or high pressure liquid chromatography (HPLC) was performed on C18 reverse phase silica, for example on a Dynamax C-18 60 Å preparative reversed-phase column;

(iv) yields, where present, are not necessarily the maximum attainable;

(v) in general, the structures of the end-products of the Formula (I) were confirmed by nuclear magnetic resonance (NMR) and/or mass spectral techniques; fast-atom bombardment (FAB) mass spectral data were obtained using a Platform spectrometer and, where appropriate, either positive ion data or negative ion data were collected; NMR chemical shift values were measured on the delta scale [proton magnetic resonance spectra were determined using a Jeol JNM EX 400 spectrometer operating at a field strength of 400 MHz, Varian Gemini 2000 spectrometer operating at a field strength of 300 MHz or a Bruker AM300 spectrometer operating at a field strength of 300 MHz;

(vi) intermediates were not generally fully characterised and purity was assessed by thin layer chromatographic, HPLC, infra-red (IR) and/or NMR analysis;

(vii) melting points are uncorrected and were determined using a Mettler SP62 automatic melting point apparatus or an oil-bath apparatus; melting points for the end-products of the formula (I) were determined after crystallisation from a conventional organic solvent such as ethanol, methanol, acetone, ether or hexane, alone or in admixture;

(viii) the following abbreviations have been used:—
DMF N,N-dimethylformamide
DCM dichloromethane
DMSO dimethylsulphoxide
THF tetrahydrofuran
DMTMM 4-(4,6-dimethoxy-1,3,5-triazinyl-2-yl)-4-methylmorpholinium chloride (see Kunishima, M., Kawachi, C., Morita, J., Terao, K., Iwasaki, F., Tani, S., Tetrahedron, 1999, 55, 13159-13170).

### EXAMPLE 1

### Preparation of N-(2-aminophenyl)-4-{5-[(isopropylamino)methyl]-1,3-thiazol-2-yl}benzamide

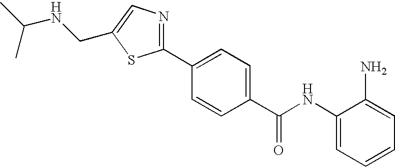

tert-Butyl [2-({4-[5-(hydroxymethyl)-1,3-thiazol-2-yl]benzoyl}amino)phenyl]carbamate (prepared as described in Method 1 below; 2.50 g, 5.88 mmol) was suspended in chloroform (110 ml) with a catalytic amount of pyridine (3 drops). Thionyl chloride (730 μl, 10.00 mmol) in chloroform (20 ml) was added dropwise and the resulting solution stirred at ambient temperature. After 2 hours, 2-methyl-2-propanol (2.50 g, 29.4 mmol) in chloroform (10 ml) was added and the solution stirred at ambient temperature for 1 hour. The mixture was evaporated under reduced pressure and dried under vacuum. The resultant solid was dissolved in DMF (30 ml) and isopropylamine (15 ml, 176.4 mmol) added. The solution was heated to 40° C. for 2 hours. The cooled solution was evaporated under reduced pressure and the residue diluted with water and a solution of saturated aqueous sodium bicarbonate. The resulting precipitate was filtered, washed with water and dried under vacuum. The solid was suspended in DCM (30 ml), trifluoroacetic acid (15 ml) added then stirred at ambient temperature for 5 hours before being absorbed onto an SCX-2 column. The column was washed with methanol (2 column volumes), then the product eluted with a 2M solution of ammonia in methanol (2 column volumes). Concentration of the ammonia/methanol solution under reduced pressure gave the crude product which was purified by reverse phase HPLC, eluting with 20 to 25% methanol in water with an acid modifier, to give the title compound (1.06 g, 49%); NMR Spectrum: (DMSO-d₆) 1.03 (d, 6H), 2.26 (br s, 1H), 2.79 (m, 1H), 3.97 (s, 2H), 4.92 (br s, 2H), 6.61 (m, 1H), 6.80 (d, 1H), 6.99 (m, 1H), 7.19 (d, 1H), 7.80 (s, 1H), 8.03 (d, 2H), 8.09 (d, 2H), 9.74 (s, 1H); Mass Spectrum: M+H⁺ 367.

### EXAMPLE 2

### Preparation of N-(2-aminophenyl)-4-{5-[(ethylamino)methyl]-1,3-thiazol-2-yl}benzamide

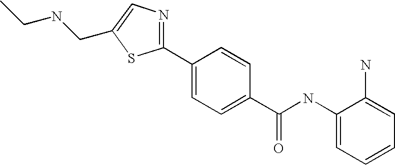

1. Chlorination Route tert-Butyl [2-({4-[5-(hydroxymethyl)-1,3-thiazol-2-yl]benzoyl}amino)phenyl]carbamate (prepared as described in Method 1 below; 1.93 g, 4.54 mmol) was suspended in chloroform (100 ml) with a catalytic amount of pyridine (3 drops). Thionyl chloride (563 μl, 7.72 mmol) in chloroform (5 ml) was added dropwise and the resulting solution stirred at ambient temperature. After 2 hours, 2-methyl-2-propanol (1.91 g, 22.7 mmol) in chloroform (5 ml) was added and the solution stirred at ambient temperature for 1 hour. The mixture was evaporated under reduced pressure and dried under vacuum. The resultant solid was dissolved in DMF (30 ml) and a 2M solution of ethylamine in THF (70 ml, 136.2 mmol) was added and the solution heated to 50° C. for 2 hours. The cooled solution was evaporated under reduced pressure then the residue dissolved in DCM/methanol (30 ml) and absorbed onto an SCX-2 column. This was washed with methanol (2 column volumes) and the product was eluted with a 2M solution of ammonia in methanol (2 column volumes). The ammonia/methanol solution was evaporated to dryness and the residue re-dissolved in DCM (30 ml). Trifluoroacetic acid (15 ml) was then added and stirred at ambient temperature for 2 hours before being absorbed onto an SCX-2 column. The column was then washed with methanol

(2 column volumes) and eluted with a 2M solution of ammonia in methanol (2 column volumes) then the ammonia/methanol solution concentrated under reduced pressure. The residue was purified by flash column chromatography, eluting with 5-10% methanol in DCM, to give the title compound (1.12 g, 70%); NMR Spectrum: (DMSO-d₆) 1.05 (t, 3H), 2.39 (br s, 1H), 2.59 (q, 2H), 3.96 (d, 2H), 4.92 (br s, 2H), 6.61 (m, 1H), 6.80 (d, 1H), 6.99 (m, 1H), 7.19 (d, 1H), 7.79 (s, 1H), 8.03 (d, 2H), 8.09 (d, 2H), 9.74 (s, 1H); Mass Spectrum: M+H⁺ 353.

### 2. Mesylation Route

A 4M solution of hydrogen chloride in 1,4-dioxane (45 ml, 180 mmol) was added to an ice bath cooled suspension of tert-butyl {2-[(4-{5′-[(ethylamino)methyl]-1,3-thiazol-2-yl}benzoyl)amino]phenyl}carbamate (prepared as described in Method 7 below; 3.9 g, 8.62 mmol) in methanol (20 ml). The reaction mixture was allowed to stir for 5 hours below 30° C. before evaporation. The resultant solid residue was dissolved in water (250 ml) and washed with ethyl acetate before basifying to pH 9 by dropwise addition of 1M aqueous sodium hydroxide solution. The resultant precipitate was collected by suction filtration and dried in a vacuum oven for 16 hours to afford the title compound (2.60 g, 86%); NMR Spectrum: (DMSO-d₆) 1.10 (t, 3H), 2.72 (q, 2H), 4.12 (s, 2H), 4.92 (br s, 2H), 6.62 (m, 1H), 6.80 (d, 1H), 6.99 (m, 1H), 7.19 (d, 1H), 7.88 (s, 1H), 8.04 (d, 2H), 811 (d, 2H), 9.76 (s, 1H); Mass Spectrum: M+H⁺ 353.

### Method 1

### Preparation of tert-butyl [2-({4-[5-(hydroxymethyl)-1,3-thiazol-2-yl]benzoyl}amino)phenyl]carbamate

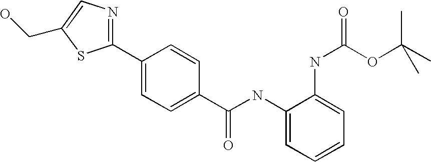

2-Chloro-4-6-dimethoxy-1,3,5-triazine (4.08 g, 23.24 mmol) was suspended in DMF (70 ml) and cooled to 5° C. under an atmosphere of nitrogen. 4-Methylmorpholine (2.56 ml, 23.24 mmol) was then added dropwise and the solution stirred at 5° C. for 10 minutes to give a suspension of DMTMM (4-(4,6-dimethoxy-1,3,5-triazinyl-2-yl)-4-methylmorpholinium chloride—see Kunishima, M., Kawachi, C., Morita, J., Terao, K., Iwasaki, F., Tani, S., Tetrahedron, 1999, 55, 13159-13170). To this suspension was added a solution of 4-[5-(hydroxymethyl)-1,3-thiazol-2-yl]benzoic acid (AZ12379701) (prepared as described in Method 2, 4.55 g, 19.37 mmol) and 1-(N-tert-butoxycarbonylamino)-2-aminobenzene (prepared according to the literature method described in Seto, C, T.; Mathias, J. P.; Whitesides, G. M.; J. Am. Chem. Soc., 1993, 115, 1321-1329; 4.84 g, 23.24 mmol) in DMF (70 ml) and the solution stirred at ambient temperature for 18 hours. A further batch of DMTMM was prepared from 2-chloro-4-6-dimethoxy-1,3,5-triazine (4.08 g, 23.24 mmol), DMF (70 ml) and 4-methylmorpholine (2.56 ml, 23.24 mmol) as described above, which was added to the reaction mixture and stirred at ambient temperature for a further 2 hours. The reaction mixture was evaporated under reduced pressure and the residue partitioned between DCM (200 ml) and water (200 ml). The DCM layer was washed with saturated aqueous sodium bicarbonate, 2M aqueous hydrochloric acid and brine, then dried over magnesium sulphate, filtered and evaporated under reduced pressure. The resultant oily residue was triturated with DCM (100 ml) and the solid filtered to give the title compound (4.86 g, 59%); NMR Spectrum: (DMSO-d₆) 1.46 (s, 9H), 4.75 (d, 2H), 5.66 (t, 1H), 7.19 (m, 2H), 7.57 (m, 2H), 7.82 (s, 1H), 8.08 (s, 4H), 8.67 (br s, 1H), 9.92 (br s, 1H); Mass Spectrum: M+H⁺ 426.

### Method 2

### Preparation of 4-[5-(hydroxymethyl)-1,3-thiazol-2-yl]benzoic acid (AZ12379701)

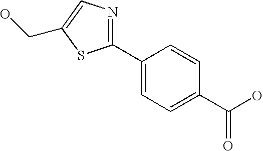

Methyl 4-{5-[(benzyloxy)methyl]-1,3-thiazol-2-yl}benzoate (prepared as described in Method 3 below; 22.15 g, 65.34 mmol) was suspended in water (200 ml) and a solution of 37% hydrochloric acid in water (200 ml) added. The reaction mixture was heated to 90° C. for 18 hours. The solution was cooled to ambient temperature and the precipitate collected by filtration. The filtrate was neutralized to pH 4 with 10M aqueous sodium hydroxide and the precipitate collected. The combined precipitates were then washed with water and dried under vacuum to give the title compound (15.99 g, quantitative) which was carried through the next step; Mass Spectrum: M+H⁺ 236.

### Method 3

### Preparation of methyl 4-{5-[(benzyloxy)methyl]-1,3-thiazol-2-yl}benzoate

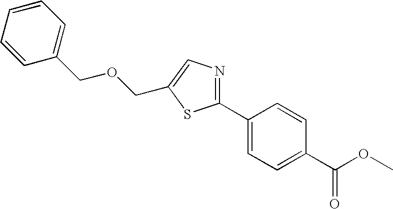

Methyl 4-({[3-(benzyloxy)-2-oxopropyl]amino}carbonyl)benzoate (prepared as described in Method 4 below, 12 g, 35.19 mmol) and Lawessons reagent (14.23 g, 35.2 mmol) were suspended in 1,4-dioxane (240 ml) and heated to 100° C. for 30 minutes. The reaction mixture was then concentrated under reduced pressure and purified by flash column chromatography, eluting with 10% ethyl acetate in isohexane, to give the title compound (7.04 g, 59%); NMR Spectrum: (DMSO-d₆) 3.89 (s, 3H), 4.59 (s, 2H), 4.83 (s, 2H), 7.32 (m, 1H), 7.37 (m, 4H), 7.95 (s, 1H), 8.08 (m, 4H); Mass Spectrum: M+H⁺ 340.

### Method 4

### Preparation of methyl 4-({[3-(benzyloxy)-2-oxopropyl]amino}carbonyl)benzoate

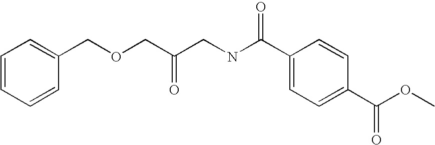

Methyl 4-({[3-(benzyloxy)-2-hydroxypropyl]amino}carbonyl)benzoate (prepared as described in Method 5 below, 25 g, 72.88 mmol) was dissolved in DCM (200 ml) and a solution of Dess-Martin periodinane (37 g, 87.5 mmol) in DCM (300 ml) was added over a 20 minute period. The reaction mixture was left stirring at ambient temperature for 18 hours. The solution was then diluted with DCM (500 ml) and washed with 1M aqueous sodium hydroxide (×3), water and brine then dried over magnesium sulfate, filtered and evaporated under reduced pressure to give the title compound (21.67 g, 87%); NMR Spectrum: (DMSO-d₆) 3.90 (s, 3H), 4.21 (d, 2H), 4.32 (s, 2H), 4.57 (s, 2H), 7.32 (m, 1H), 7.38 (m, 4H), 7.99 (d, 2H), 8.07 (d, 2H), 8.95 (t, 1H); Mass Spectrum: M+H⁺ 342.

### Method 5

### Preparation of methyl 4-({[3-(benzyloxy)-2-hydroxypropyl]amino}carbonyl)benzoate

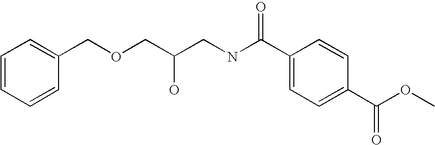

1-(Benzyloxy)-3-{[(1E)-phenylmethylene]amino}propan-2-ol (prepared as described in Method 6 below, 25 g, 92.82 mmol) was dissolved in chloroform (75 ml) and pyridine (7.57 ml, 92.8 mmol) added. The solution was cooled to −40° C. under a nitrogen atmosphere and methyl 4-chlorocarbonylbenzoate (18.43 g, 92.8 mmol) in DCM (75 ml) was added dropwise over a 30 minute period. The solution was stirred at −40° C. for a further 30 minutes then stirred at ambient temperature for 18 hours. The DCM was evaporated under reduced pressure and a solution of 37% hydrochloric acid in water (93 ml) added, then stirred for 10 minutes. The mixture was diluted with water (225 ml) and isohexane (225 ml), covered and placed in the refrigerator. After 3 hours the precipitate was filtered, washed with water, then added to a solution of saturated aqueous sodium carbonate. The suspension was placed in a sonicated for 10 minutes then filtered, washed with water, then isohexane and dried, to give the title compound (28.30 g, 89%); NMR Spectrum: (DMSO-d₆) 3.25 (m, 1H), 3.43 (m, 3H), 3.87 (br m, 4H), 4.51 (s, 2H), 5.00 (br s, 1H), 7.29 (m, 1H), 7.34 (m, 4H), 7.96 (d, 2H), 8.03 (d, 2H), 8.57 (t, 1H); Mass Spectrum: M+H⁺ 344.

### Method 6

### Preparation of 1-(benzyloxy)-3-{([(1E)-phenylmethylene]amino}propan-2-ol

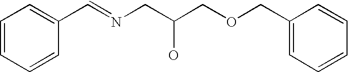

Benzaldehyde (44.8 g, 422 mmol) and 28% ammonium hydroxide solution (42.2 ml) were stirred in ethanol (150 ml) at ambient temperature for 10 minutes. Benzyl glycidyl ether (69.3 g, 422 mmol) in ethanol (70 ml) was added over a period of an hour and the solution was stirred at ambient temperature for 18 hours, then at reflux for 30 minutes. The cooled solution was evaporated under reduced pressure and the residues were added to ice/water (45 ml) and cooled in an ice bath for 2 hours. The resulting solid was filtered, dried under vacuum then recrystallised from isohexane, to give the title compound (69.80 g, 61%); NMR Spectrum: (DMSO-d₆) 3.49 (br m, 3H), 3.74 (m, 1H), 3.93 (m, 1H), 4.53 (s, 2H), 4.81 (d, 1H), 7.29 (m, 1H), 7.35 (m, 4H), 7.45 (m, 3H), 7.73 (m, 2H), 8.31 (s, 1H).

### Method 7

### Preparation of tert-butyl {2-[(4-{5-[(ethylamino)methyl]-1,3-thiazol-2-yl}benzoyl)amino]phenyl}carbamate

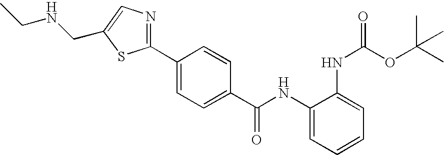

To a suspension of tert-butyl [2-({4-[5-(hydroxymethyl)-1,3-thiazol-2-yl]benzoyl}amino)phenyl]carbamate (prepared as described in Method 8 below; 7.6 g, 17.86 mmol) in DCM (200 ml), was added, methanesulfonyl chloride (3.0 ml, 38.8 mmol). Triethylamine (5.3 ml, 39.1 mmol) was then added dropwise over 10 minutes, so as to maintain the internal temperature below 30° C. The resultant solution was stirred at ambient temperature for 30 minutes, before being cooled to 10° C. and treated dropwise with a 2.0M solution of ethylamine in tetrahydrofuran (90 ml, 180 mmol). The reaction mixture was stirred and allowed to warm to room temperature for 22 hours before evaporation to dryness. The residue was partitioned between DCM and water and organic layer separated. The aqueous was re-extracted with further portions of DCM (×2) and combined organic extracts washed with brine, dried over magnesium sulphate and evaporated to dryness to give a brown gum, which was purified by flash chromatography on silica eluting with a rising gradient of 0-10% methanol in DCM to afford the title compound (4.0 g, 49%); NMR Spectrum: (DMSO-d₆) 1.05 (t, 3H), 1.46 (s, 9H), 2.39 (br m, 1H), 2.59 (q, 2H), 3.96 (s, 2H), 7.19 (m, 2H), 7.56 (m, 2H), 7.80 (s, 1H), 8.07 (s, 4H), 8.67 (s, 1H), 9.91 (s, 1H); Mass Spectrum: M+H⁺ 453.

### Method 8

### Preparation of tert-butyl [2-({4-[5-(hydroxymethyl)-1,3-thiazol-2-yl]benzoyl}amino)phenyl]carbamate

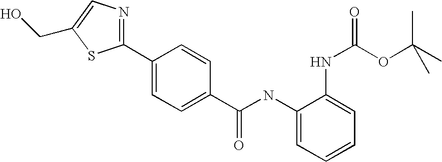

A solution of sodium borohydride (2.30 g, 60.9 mmol) in water (50 ml) was added dropwise to a suspension of tert-butyl(2-{[4-(5-formyl-1,3-thiazol-2-1)benzoyl]amino}phenyl)carbamate (prepared as described in Method 9 below; 7.85 g, 18.5 mmol) in methanol (500 ml), which is cooled in an ice bath (internal temp 10° C.). The reaction mixture was then allowed to warm to room temperature and stirred for 18 hours before addition of a further portion of sodium borohydride (250 mg, 6.61 mmol). Stirring was continued for a further 3 hours and the reaction mixture then filtered and the filtrate evaporated to dryness. The residue was partitioned between DCM and saturated aqueous sodium bicarbonate solution. The insoluble material, which precipitated at the phase boundary, was collected by suction filtration. The filtrate was separated and the organic layer washed with water and brine, dried over magnesium sulfate and evaporated to dryness to give a beige solid. The two solids obtained were combined and dissolved in methanol and re-evaporated to give the title compound (7.6 g, 97%); NMR Spectrum: (DMSO-d₆) 1.46 (s, 9H), 4.75 (s, 2H), 7.15 (m, 2H), 7.58 (m, 2H), 7.82 (s, 1H), 8.07 (m, 4H); Mass Spectrum: MH⁺ 426

### Method 9

### Preparation of tert-butyl (2-{[4-(5-formyl-1,3-thiazol-2-yl)benzoyl]amino}phenyl)carbamate

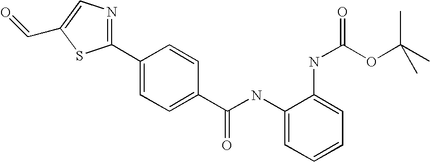

To a 3-neck, 1 litre round bottomed flask, equipped with an overhead stirrer, was added N-(2-tert-butoxycarbonylaminophenyl)-4-(4,4,5,5-tetramethyl-1,3,2-dioxaborolan-2-yl)benzamide (prepared as described in International Patent Publication Number WO03/087057, Method 13, page 60; 10.2 g, 23.3 mmol) and 1,2-dimethoxyethane (300 ml). To this solution was added a solution of 2-chloro-1,3-thiazole-5-carbaldehyde (4.5 g, 30.5 mmol) in 1,2-dimethoxyethane (100 ml). Dichlorodiphenylphospinoferrocenyl palladium (II) (1.2 g, 1.5 mmol) was then added followed by dropwise addition, over 10 minutes, of saturated aqueous sodium bicarbonate solution (200 ml). The reaction mixture was stirred for 16 hours before cooling to ambient temperature. The solid precipitate was then collected by suction filtration, washed with MeOH and water and dried under vacuum for 18 hours to afford the title compound (6.7 g, 68%); NMR Spectrum: (DMSO-d₆) 1.46 (s, 9H), 7.19 (m, 2H), 7.57 (m, 2H), 8.13 (d, 2H), 8.25 (d, 2H), 8.68 (br s, 1H), 8.84 (s, 1H), 9.97 (br s, 1H), 10.13 (s, 1H); Mass Spectrum: MH⁺-Boc 324.

1. A compound of formula (I): 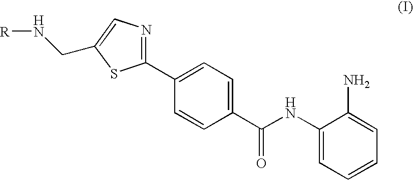
wherein R is ethyl or isopropyl;
or a pharmaceutically acceptable salt or pro-drug form thereof.

2. N-(2-aminophenyl)-4-{5-[(ethylamino)methyl]-1,3-thiazol-2-yl}benzamide.

3. N-(2-aminophenyl)-4-{5-[(isopropylamino)methyl]-1,3-thiazol-2-yl}benzamide.
